(19) United States
(12) Patent Application Publication
(10) Pub. No.: US 2025/0312599 A1
(43) Pub. Date: Oct. 9, 2025

(54) MRI-Safety and Force Optimized Implant Magnet System

(71) Applicant: MED-EL ELEKTROMEDIZINISCHE GERAETE GMBH, Innsbruck (AT)

(72) Inventor: Thomas Wilhelm Eigentler, Sistrans (AT)

(21) Appl. No.: 19/244,906

(22) Filed: Jun. 20, 2025

Related U.S. Application Data
(63) Continuation of application No. 18/416,463, filed on Jan. 18, 2024, now abandoned.
Application No. 18/416,463 is a continuation of application No. 17/982,434, filed on Nov. 7, 2022, now abandoned.
Application No. 17/982,434 is a continuation of application No. 16/607,798, filed on Oct. 24, 2019, now abandoned.
(60) Provisional application No. 62/488,932, filed on Apr. 24, 2017.

(51) Int. Cl. A61N 1/36; A61N 1/372; H04R 25/00

(52) U.S. Cl. CPC A61N 1/36038; A61N 1/372; H04R 25/70; H04R 2225/67

(57) ABSTRACT

A magnet arrangement for an implantable medical device is described. An implant magnet has a modified disc shape and is capable of responding to an external magnetic field by rotating about a primary center rotation axis. The implant magnet shape has at least one cross-sectional view, the cylindrical diameter corresponds to a horizontal coordinate axis, the center symmetry axis corresponds to a vertical coordinate axis, the height between the end surfaces is greatest at the center symmetry axis, and the height between the end surfaces progressively decreases from the center symmetry axis along the cylindrical diameter towards the outer circumference to define a secondary deflection angle with respect to the horizontal coordinate axis so that the implant magnet is capable of responding to the external magnetic field by deflecting within the secondary deflection angle about a secondary deflection axis defined by a cylinder diameter normal to the cross-sectional view.

This application is a continuation of U.S. patent application Ser. No. 18/416,463, filed Jan. 18, 2024, which is a continuation of U.S. patent application Ser. No. 17/982,434, filed Nov. 7, 2022, which is a continuation of U.S. patent application Ser. No. 16/607,798, filed Oct. 24, 2019, which is a national phase entry of International Patent Application No. PCT/US2018/028785, filed Apr. 23, 2018, which claims priority from U.S. Provisional Patent Application No. 62/488,932, filed Apr. 24, 2017, the disclosures of which are incorporated herein by reference in their entirety.

### FIELD OF THE INVENTION

The present invention relates to implantable hearing devices such as cochlear implants, and specifically, to implantable magnets in such devices.

### BACKGROUND ART

Some hearing implants such as Middle Ear Implants (MEI's) and Cochlear Implants (CI's) employ cooperating attachment magnets located in the implant and the external part to hold the external part in place over the implant. For example, as shown in FIG. 1, a typical hearing implant system may include an external transmitter housing 101 containing transmitting coils 107 and an external attachment magnet 105. The external attachment magnet 105 has a conventional cylindrical disc-shape and a north-south magnetic dipole having an axis that is perpendicular to the skin of the patient as shown. Implanted under the patient's skin is a corresponding receiver assembly 102 having similar receiving coils 108 and an implant magnet 106. The implant magnet 106 also has a cylindrical disc-shape and a north-south magnetic dipole having a magnetic axis that is perpendicular to the skin of the patient as shown. The internal receiver housing 102 is surgically implanted and fixed in place within the patient's body. The external transmitter housing 101 is placed in proper position over the skin covering the internal receiver assembly 102 and held in place by interaction between the magnets 105 and 106 thus, the internal magnetic field lines and the external magnetic field lines. Rf signals from the transmitter coils 107 couple data and/or power to the receiving coil 108 which is in communication with an implanted processor module (not shown).

One problem with the typical hearing implant, as shown in FIG. 1, arises when the patient undergoes Magnetic Resonance Imaging (MRI) examination. Interactions occur between the implant magnet and the applied external magnetic field for the MRI. As shown in FIG. 2, the direction of the magnetic dipole \vec{m} of the implant magnet 202 is essentially B from perpendicular to the skin of the patient. In this example, the strong static magnetic field the MRI creates a torque \vec{T}=\vec{m}×\vec{B} on the internal magnet 202, which may displace the internal magnet 202 or the whole implant housing 201 out of proper position. Among other things, this may damage the implant or the adjacent tissue of the patient. In addition, the external magnetic field \vec{B} from the MRI may reduce, remove or invert the magnetic dipole \vec{m} of the implant magnet 202 so that it may no longer be able or strong enough to hold the external transmitter housing in proper position. Torque and forces acting on the implant magnet and demagnetization of the implant magnet is especially an issue with MRI field strengths exceeding 1.5 Tesla.

Thus, for existing implant systems with magnet arrangements, it is common to either not permit MRI, or at most limit use of MRI to lower field strengths. Other existing solutions include use of a surgically removable magnets, spherical implant magnets (e.g. U.S. Pat. No. 7,566,296), and various ring magnet designs (e.g., U.S. Patent Publication 2011/0022120).

U.S. Pat. No. 8,634,909 describes an implant magnet having a diametrical magnetization, where the magnetic axis is parallel to the end surfaces of a disc shaped implant magnet—that is, perpendicular to the conventional magnetic axis of a disc-shaped implant magnet. The magnet is then held in a receptacle that allows the magnet to rotate about its center axis in response to an external magnetic field such as from an MRI to realign and avoid creating torque. But this rotation is only possible around a single axis, the central axis.

FIG. 3 shows the head of a patient with bilateral hearing implants 301 having such an implant magnet in the presence of a typical MRI scanning magnetic field B₀, which is aligned along the longitudinal axis of the patient. The magnetization axis of the hearing implants 301 is angled with respect to the magnetic field \vec{B} at some relative angle α_{B} as shown in FIG. 3, which can create an undesirable torque on the hearing implants 301. This relative angle α_{B} is dependent on the individual patient's anatomy and the exact implant position, for example on the skull of the patient.

FIG. 4 shows in greater detail the geometry of an implant magnet 401 with a magnetic dipole \vec{m} that is parallel to the skin, and an MRI scanning magnetic field \vec{B} aligned along the longitudinal symmetry axis. The cylindrical disc shape of the implant magnet 401 has a height h and a diameter Ød. Depending on the specific orientation of the implant within the patient, there will be a relative angle α_{B} between the direction of the magnetic dipole \vec{m} of the implant magnet 401 and the static magnetic field \vec{B}. The relative angle α_{B} also remains when implant magnet 401 is rotatable about its cylindrical axis 402, as for example described in U.S. Pat. No. 8,634,909. This relative angle α_{B} leads to a torque force on the implant magnet 401, where the torque \vec{T}=\vec{m}×\vec{B}, and the force at the circumference of the stiff structure is \vec{F}=\vec{T}/D, where D is the distance or diameter of the stiff structure surrounding the implant magnet 401.

### SUMMARY OF THE INVENTION

Embodiments of the present invention are directed to a magnet arrangement for a hearing implant device. An implantable magnet has a modified disc shape with a primary center rotation axis, a cylindrical height, a diameter, an outer circumference and opposing end surfaces. The implant magnet shape has at least one cross section view in which the primary center rotating axis is defined where the height of the magnet system is greatest and an axis normal to the cross section view is defining the secondary deflection axis. This magnet shape is capable of responding to an external magnetic field by rotating about the primary center rotation axis. The implant magnet shape has at least one cross-sectional view in which the cylindrical diameter corresponds to a horizontal coordinate axis, the primary center rotation axis corresponds to a vertical coordinate axis, and the height between the end surfaces is greatest. The height between the end surfaces progressively decreases from the primary center rotation axis along the cylindrical diameter towards the outer circumference to define a secondary deflection angle with respect to the horizontal coordinate axis so that the implant magnet is capable of responding to the external magnetic field by deflecting within the secondary deflection angle about a secondary deflection axis defined by a cylinder diameter normal to the at least one cross-sectional view.

In further specific embodiments, there may also be a magnet housing enclosing a cylindrical shaped interior volume that contains the implant magnet. The implant magnet then is configured to securely fit within the interior volume so as to allow free alignment to an external magnetic field about the primary rotating axis as is limited partial rotation about the secondary deflection axis. In such embodiments, the interior volume may contain a damper oil which surrounds the implant magnet and/or at least one ferromagnetic domain which enabled a magnetic fixation of the implant magnet inside the embodiment. The implant magnet may include one or more low-friction contact surfaces configured to connect the implant magnet to the magnet housing.

The at least one cross-sectional view may be exactly one cross-sectional view, or it may be every cross-sectional view in which the cylindrical diameter corresponds to a horizontal coordinate axis and the primary center rotation axis corresponds to a vertical coordinate axis.

Embodiments of the present invention also include a hearing implant system containing a magnet arrangement according to any of the foregoing.

### BRIEF DESCRIPTION OF THE DRAWINGS

FIG. 1 shows portions of a typical cochlear implant system and the magnetic interaction between the implant magnet and the external holding magnet.

FIG. 2 illustrates the force interactions that can occur between an implant magnet and the applied external magnetic field for an MRI system.

FIG. 3 the head of a patient with bilateral cochlear implants in the presence of a typical MRI scanning magnetic field.

FIG. 4 shows geometry of an implant magnet with a magnetic dipole parallel to the skin and an MRI scanning magnetic field.

FIG. 5 shows cross-sectional view geometry of a modified disc-shaped implant magnet according to an embodiment of the present invention.

FIG. 6 shows a cross-sectional view of an implant magnet enclosed within a magnet housing.

FIG. 7 shows geometry of an implant magnet arrangement according to FIG. 6 in an MRI scanning magnetic field.

FIGS. 8A-8B show elevated perspective views of a rotationally symmetric and a non-rotationally symmetric implant magnet according to embodiments of the present invention.

FIG. 9 shows a cross-sectional view of an implant magnet arrangement with friction-reducing surfaces according to an embodiment of the present invention.

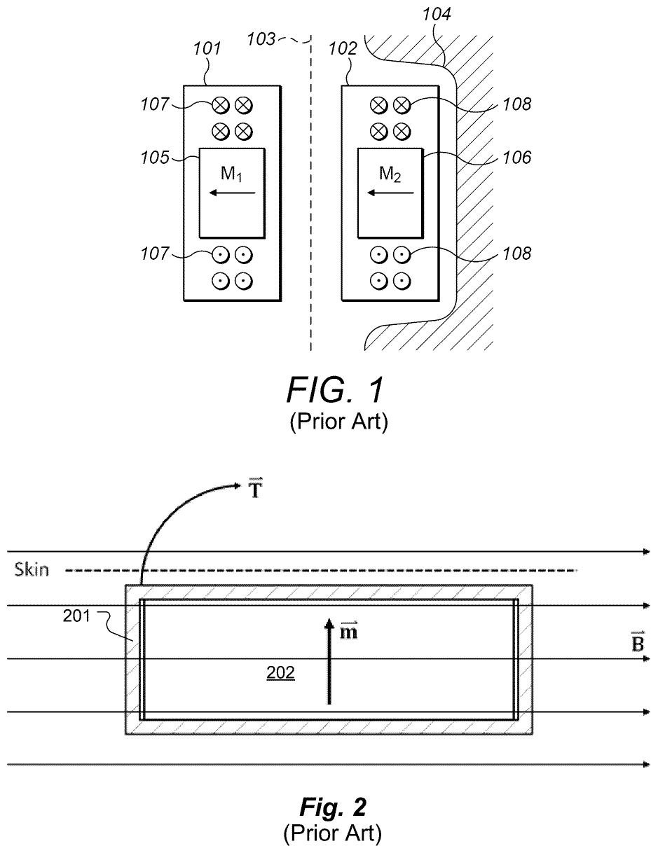

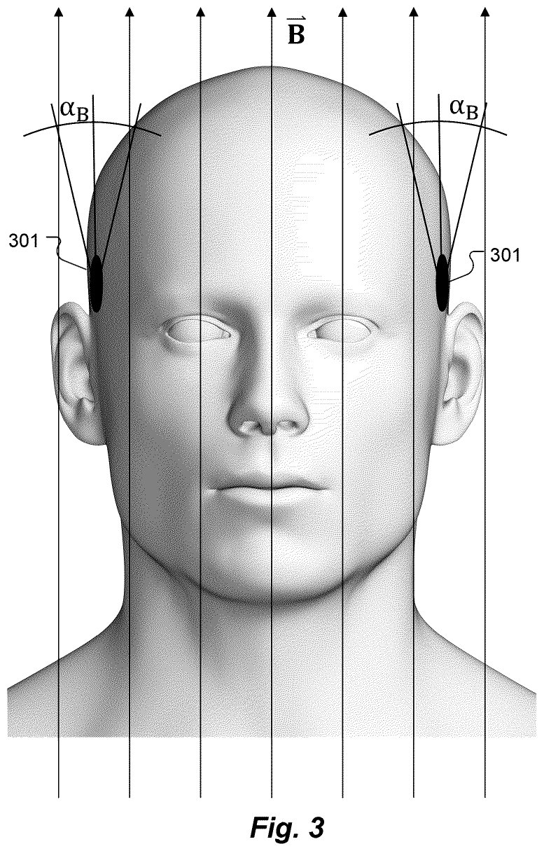

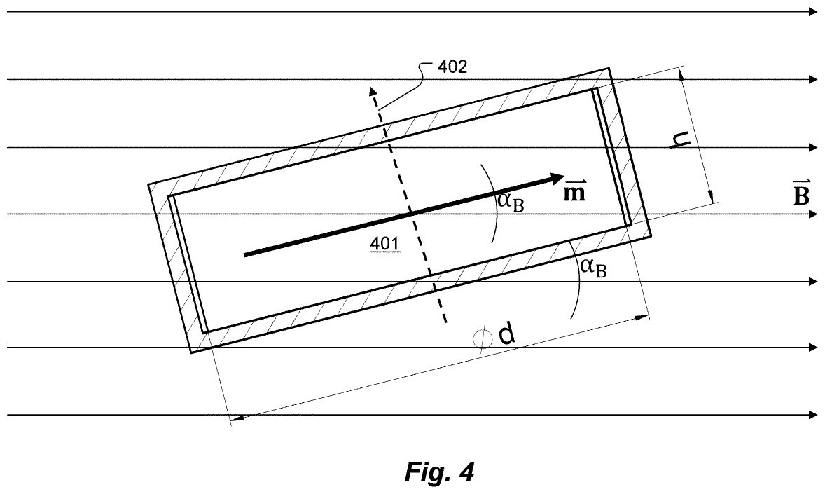

### DETAILED DESCRIPTION OF SPECIFIC EMBODIMENTS

Embodiments of the present invention are directed to an improved implant magnet that can achieve a lower mechanical force during an MRI for a given magnetization or magnet strength. The inventive implant magnet has a limited deflection rotation about a secondary deflection axis to reduce the torque created by the static magnetic field \vec{B} in the MRI-scanner. This, in turn, allows use of a stronger implant magnet with the same mechanical torque during MRI.

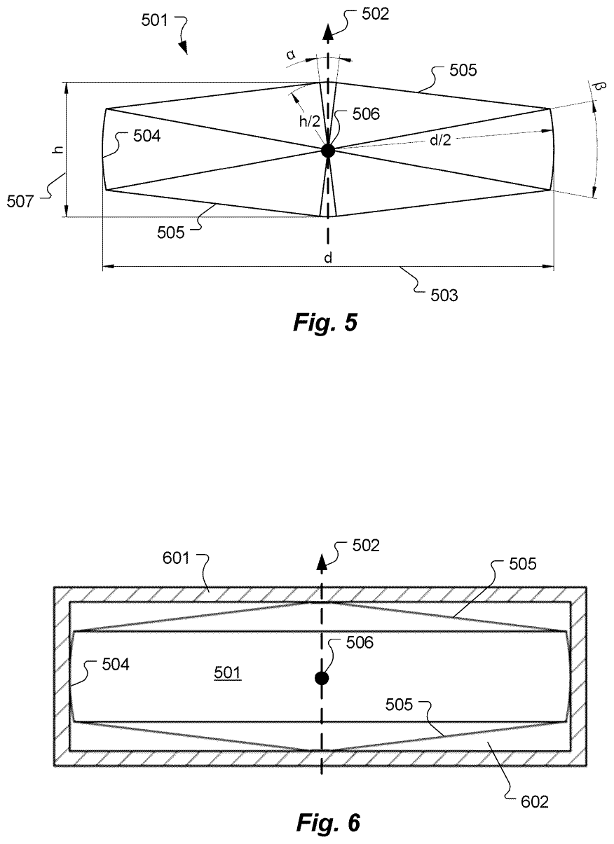

FIG. 5 shows the cross-sectional view geometry of an implant magnet 501 according to one embodiment of the present invention, with a center rotation axis 502, a cylindrical height 507 and diameter 503, an outer circumference 504, and opposing end surfaces 505. The implant magnet 501 is capable of responding to an external magnetic field \vec{B} by rotating about the center rotation axis 502. And the shape of the implant magnet 501 has at least one cross-sectional view as shown in FIG. 5 where the cylindrical diameter 503 corresponds to a horizontal coordinate axis, the primary center rotation axis 502 corresponds to a vertical coordinate axis. The height 507 of the implant magnet 501 between the end surfaces 505 is greatest at the primary center rotation axis 502 and progressively decreases from the primary center rotation axis 502 along the cylindrical diameter 503 towards the outer circumference 504.

FIG. 6 shows a cross-sectional view of a further specific embodiment with a magnet housing 601 that encloses a cylindrical shaped interior volume 602 that contains the implant magnet 501. The implant magnet 501 is configured to securely fit within the interior volume 602 so as to be freely rotatable about the primary center rotation axis 502 and the secondary deflection axis 506. In such embodiments, the interior volume 602 may contain a damper oil (to reduce rattler noise) which surrounds the implant magnet 501.

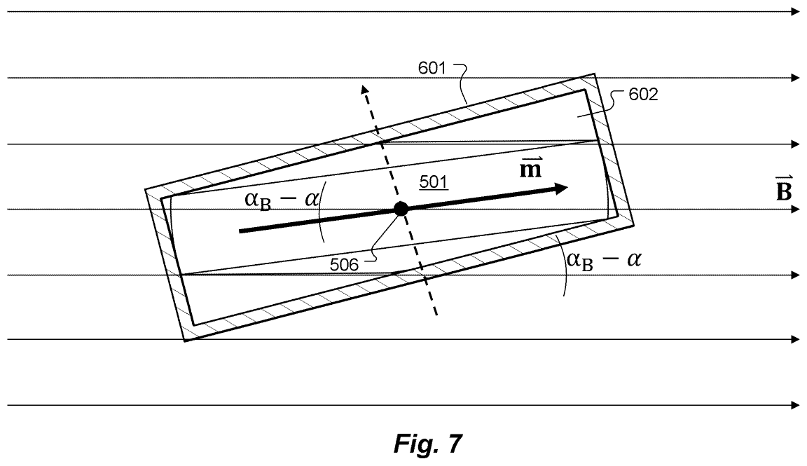

The geometry of the implant magnet 501 defines a secondary deflection angle α_{B} with respect to the horizontal coordinate axis so that the implant magnet 501 is capable of responding to the external magnetic field \vec{B}, as shown in FIG. 7, by deflecting within the secondary deflection angle α_{B} about a secondary deflection axis 506 which is normal to the at least one cross-sectional view, up until further secondary rotation is prevented by the end surfaces 505 pressing against the inner surface of the magnet housing 601 as shown in FIG. 7.

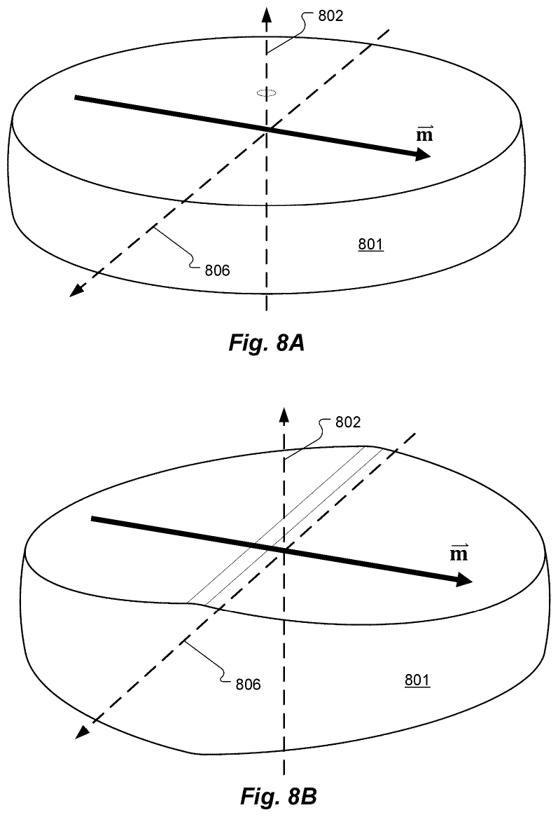

FIGS. 8A-8B show elevated perspective views of two different shape approaches to an implant magnet 801 according to an embodiment of the present invention. The implant magnet 801 shown in FIG. 8A is rotationally symmetric. The end surfaces on the top and bottom of the disc-shaped implant magnet 801 form two rounded cones centered around the primary center rotation axis 802 with a chamfer radius of half the magnet height. Every cross-sectional view through the end surfaces will be such that the height is greatest at the center of the primary center rotation axis 802 and progressively decreases radially outward towards the outer circumference. To enable a secondary deflection around a secondary deflection axis 806, the edges of the cylindrical diameter are chamfered with the radius of the half diameter. In such a rotationally symmetric implant magnet 801 the diametrical magnetization in every direction is normal to the primary rotation axis 802.

The implant magnet 801 shown in FIG. 8B is non-rotationally symmetric design with a rounded dam-shaped design on the top and bottom of the cylindrical implant magnet 801 with the radius of the chamfers the same as in the symmetric design in FIG. 8A. For such a non-rotationally symmetric shape, the direction of the magnetic dipole \vec{m} has to align normal to the secondary deflection axis 806, which is in turn parallel to the top and bottom line of the dam-shape. It will be appreciated in this embodiment, there is just a single cross-sectional view where the magnet height is greatest at the primary center rotation axis 802 and progressively decreases radially outward towards the outer circumference.

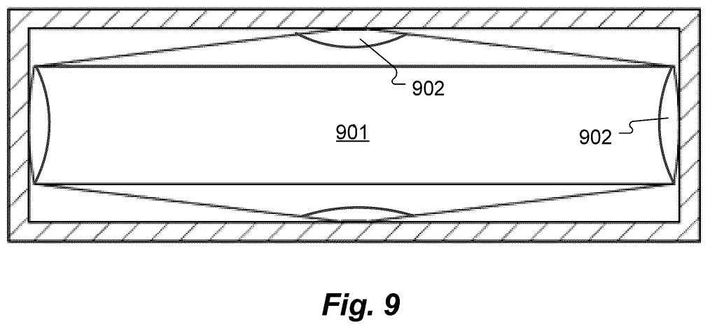

FIG. 9 shows a cross-sectional view of a further specific embodiment where the implant magnet 901 includes one or more low-friction contact surfaces 902, e.g. made of titanium, that are configured to connect the implant magnet 901 to the magnet housing; for example, at the center axis of symmetry and/or at the outer circumference.

Although various exemplary embodiments of the invention have been disclosed, it should be apparent to those skilled in the art that various changes and modifications can be made which will achieve some of the advantages of the invention without departing from the true scope of the invention.

What is claimed is:

1. A magnet arrangement for an implantable hearing device, the magnet arrangement comprising:
an implantable holding magnet having a modified disc shape with a primary center rotation axis, a cylindrical height and diameter, an outer circumference, and opposing end surfaces;
wherein the implantable holding magnet is capable of responding to an external magnetic field by rotating about the primary center rotation axis, and
wherein the implantable holding magnet shape has at least one cross-sectional view in which:
i. the cylindrical diameter corresponds to a horizontal coordinate axis,
ii. the primary center rotation axis corresponds to a vertical coordinate axis,
iii. height between the end surfaces is greatest at the primary center rotation axis, and
iv. height between the end surfaces progressively decreases from the primary center rotation axis along the cylindrical diameter towards the outer circumference to define a secondary deflection angle with respect to the horizontal coordinate axis so that the implantable holding magnet is capable of responding to the external magnetic field by deflecting within the secondary deflection angle about a secondary deflection axis defined by a cylinder diameter normal to the at least one cross-sectional view.

2. The magnet arrangement according to claim 1, further comprising:
a magnet housing enclosing a cylindrical shaped interior volume containing the implantable holding magnet, wherein the implantable holding magnet is configured to securely fit within the interior volume so as to be freely rotatable about the primary center rotating axis and about the secondary deflection axis.

3. The magnet arrangement according to claim 2, wherein the interior volume contains a damper oil which surrounds the implantable holding magnet.

4. The magnet arrangement according to claim 2, wherein the interior volume contains at least one ferromagnetic domain which surrounds the implantable holding magnet.

5. The magnet arrangement according to claim 2, wherein the implantable holding magnet includes one or more low-friction contact surfaces configured to connect the implantable holding magnet to the magnet housing.

6. The magnet arrangement according to claim 5, wherein the one or more low-friction contact surfaces are located at the center axis of symmetry.

7. The magnet arrangement according to claim 5, wherein the one or more low-friction contact surfaces are located at the outer circumference.

8. The magnet arrangement according to claim 1, wherein the at least one cross-sectional view is one cross-sectional view, which is a geometric non-rotationally symmetric design.

9. The magnet arrangement according to claim 1, wherein the at least one cross-sectional view is every cross-sectional view in which the cylindrical diameter corresponds to a horizontal coordinate axis and the primary center rotation axis corresponds to a vertical coordinate axis, which is a geometric rotationally symmetric design.

10. A hearing implant system comprising:
an implantable hearing device having a magnet arrangement, the magnet arrangement comprising:
an implantable holding magnet having a modified disc shape with a primary center rotation axis, a cylindrical height and diameter, an outer circumference, and opposing end surfaces;
wherein the implantable holding magnet is capable of responding to an external magnetic field by rotating about the primary center rotation axis, and
wherein the implantable holding magnet shape has at least one cross-sectional view in which:
i. the cylindrical diameter corresponds to a horizontal coordinate axis,
ii. the primary center rotation axis corresponds to a vertical coordinate axis,
iii. height between the end surfaces is greatest at the primary center rotation axis, and
iv. height between the end surfaces progressively decreases from the primary center rotation axis along the cylindrical diameter towards the outer circumference to define a secondary deflection angle with respect to the horizontal coordinate axis so that the implantable holding magnet is capable of responding to the external magnetic field by deflecting within the secondary deflection angle about a secondary deflection axis defined by a cylinder diameter normal to the at least one cross-sectional view; and
an external device comprising:
an external transmitter housing containing an external attachment magnet configured to interact with the implantable holding magnet to hold the external device in place over the implantable hearing device on a patient's skin.

11. The hearing implant system of claim 10, wherein the magnetic arrangement further comprises:
a magnet housing enclosing a cylindrical shaped interior volume containing the implantable holding magnet, wherein the implantable holding magnet is configured to securely fit within the interior volume so as to be freely rotatable about the primary center rotating axis and about the secondary deflection axis.

12. The hearing implant system of claim 11, wherein the interior volume contains a damper oil which surrounds the implantable holding magnet.

13. The hearing implant system of claim 11, wherein the interior volume contains at least one ferromagnetic domain which surrounds the implantable holding magnet.

14. The hearing implant system of claim 11, wherein the implantable holding magnet includes one or more low-friction contact surfaces configured to connect the implantable holding magnet to the magnet housing.

15. The hearing implant system of claim 14, wherein the one or more low-friction contact surfaces are located at the center axis of symmetry.

16. The hearing implant system of claim 14, wherein the one or more low-friction contact surfaces are located at the outer circumference.

17. The hearing implant system of claim 10, wherein the at least one cross-sectional view is one cross-sectional view, which is a geometric non-rotationally symmetric design.

18. The hearing implant system of claim 10, wherein the at least one cross-sectional view is every cross-sectional view in which the cylindrical diameter corresponds to a horizontal coordinate axis and the primary center rotation axis corresponds to a vertical coordinate axis, which is a geometric rotationally symmetric design.
